(19) United States
(12) Patent Application Publication
(10) Pub. No.: US 2023/0099672 A1
(43) Pub. Date: Mar. 30, 2023

(54) CHARGING CIRCUIT FOR A DEFIBRILLATOR

(71) Applicants: Altrix Medical, Inc., Centreville, VA (US); Defibtech, LLC, Guilford, CT (US)

(72) Inventors: Gene Glick, Stratford, CT (US); Daniel Fleck, Potomac, MD (US); David Hicks, Mystic, CT (US); Michael Hicks, Madison, CT (US); Matthew Shaker, Centreville, VA (US)

(21) Appl. No.: 17/484,528

(22) Filed: Sep. 24, 2021

(51) Int. Cl. A61N 1/39

(52) U.S. Cl. CPC A61N 1/3981; A61N 1/3912; A61N 1/3904

(57) ABSTRACT

A charging circuit for a capacitor in a defibrillator includes a control enabling a setting of a desired time to charge a capacitor to a desired voltage in the defibrillator. The charging circuit further includes a flyback charge-pump circuit comprising a switch, an energy transfer transformer, an energy storage capacitor and a control. The switch is configured to stop or allow storage of energy in a transformer. The transformer transfers the energy to the capacitor. The flyback charge-pump circuit controls a duty-cycle on the switch so that a current draw from a power source (e.g. battery) is sufficient to enable charging the capacitor to the desired voltage within the desired time set on the control.

### GOVERNMENT LICENSE RIGHTS

This invention was made with Government support under Cooperative Agreement 2026090 awarded by the National Science Foundation. The Government has certain rights to this invention.

### TECHNICAL FIELD

In the field of electric power conversion systems, wherein a single electrical source circuit is coupled to a single electrical load circuit, the operation of which is controlled by condition responsive means.

### BACKGROUND ART

At the heart of a defibrillator is a capacitor that holds a charge for a shock. The capacitor is part of a charging circuit that can be thought of as a “charge-pump.”

A basic charge pump uses a circuit with a primary side and a secondary side. The primary side has a power source (such as a battery or multiple battery cells), a switch, and a primary induction coil in the transformer. The flyback-style circuit uses a “flyback transformer” made from a primary inductor and secondary inductor wound on a common core with the winding polarity 180 degrees out of phase to store energy for later release to a capacitor. In such a scenario, the secondary side of the transformer has a switch, such as a diode that prevents current flow from the capacitor back to the transformer when current flows in the primary induction coil. The transformer transfers its energy to a capacitor on the secondary side when the primary side switch is open.

A flyback-style circuit or charge pump has, theoretically, no limit on the output voltage and the transformer isolates and protects the primary-side circuitry from the secondary-side high-voltage circuitry by way of galvanic isolation. Instead of a charge pump, it is also possible to create a charging circuit using other common methods such as step-up/down transformer coupled (non-flyback) or a capacitively-coupled charge pump. Any of these possible charging circuit designs comprise a power translator.

### SUMMARY OF INVENTION

A charging circuit for a capacitor in a defibrillator is disclosed. The charging circuit includes a control enabling a setting of a desired time for charging a capacitor in the defibrillator. The charging circuit further includes a transformer, a power source and power translator.

The transformer includes a primary-side circuit and a secondary-side circuit. The primary-side circuit includes a primary-side induction coil. The secondary-side circuit includes a secondary-side induction coil that is galvanically isolated from the primary-side circuit.

The power translator is configured to vary the power draw from the power source to meet the setting on the control of the desired time for charging the capacitor. Essentially, it is a circuit that couples a power source to the capacitor in a way that allows control of the capacitor charging time and the power consumption rate from the source.

The power translator may include a switch in the primary-side circuit. The switch is configured to stop or enable current flow from the power source to the primary-side circuit. The power translator controls the switch to stop or enable current flow from the power source to the primary-side induction coil so that an average power draw from the power source is sufficient to transfer energy to charge the capacitor within the desired time set on the control. Preferably, the power translator is configured to maintain an approximately constant average power from the power source to the power translator.

The power translator may include a flyback-style circuit configured to have no limit on output voltage from the charged capacitor.

Additional optional controls may be utilized to maintain an approximately constant voltage from the power source to the power translator; monitor the cycle-by-cycle current to the capacitor from the transformer; stop energy saturation of the transformer core by preventing the power translator from sending a charge pulse of energy to the transformer unless the cycle-by-cycle current to the capacitor is approximately zero; monitor a cycle-by-cycle current from the power source to the transformer, and to stop energy draw from the power source if an over-current is detected.

The charging circuit may include: a boost regulator configured to deliver an approximately constant voltage to the power translator; a variable resistor configured to adjust its resistance in a feedback loop to maintain an approximately constant charging current delivered to the capacitor; a power translator that varies a duty cycle of the switch to maintain a constant average current from the power source; and a pulse-width modulation chip configured with a current-sensing variable resistor and threshold detector further controlled by a current sensing circuit which determines average current and controls the current sensing variable resistor that in turn controls the pulse width modulation chip to maintain an approximately uniform average power draw from the power source by adjusting the duty cycle on the switch.

### Technical Problem

The traditional charging circuit for a defibrillator does not permit an empirically derived charging time.

Secondly, traditional flyback-type charging circuits used in many defibrillators initially saturate the transformer core from the primary side and require additional circuitry to protect against excessive current draw after that saturation. The result of such designs is to utilize variable duty cycles to perform the transfer of energy. The disadvantage of such non-deterministic duty cycles is that the transformer might not have enough time to release all the stored energy to the capacitor, resulting in additional circuitry and methods to monitor energy transfer to the transformer and manage power source draw.

### Solution to Problem

The solution is a charging circuit that provides a settable constant average power draw on the power source. Then, given a known target voltage, this charging circuit provides for a settable charging time.

The solution is a charging circuit that eliminates or substantially reduces transformer saturation.

The solution is a charging circuit that enables optimal power source management (e.g., being able to manage weak batteries).

### Advantageous Effects of Invention

The disclosed charging circuit allows for a predictable charging time for the capacitor by enabling a settable charging current. The charging circuit works by providing a constant average current and constant voltage from a boost regulator going to the flyback charge pump circuit. It provides constant average current by controlling the duty-cycle on the switch of the flyback charge-pump circuit (a mechanism to store energy in a transformer and release it).

The disclosed charging circuit monitors the current on the secondary side of the charge-pump on every charging cycle where a charging cycle is defined as one full iteration that includes the switch being open and closed. The disclosed charging circuit will not allow the next charging cycle to begin until the transformer has released all its stored energy and the current to the capacitor from the secondary has dropped to nearly zero. This assures that the energy stored in the transformer has been consumed. Given a fixed frequency, the operation may include cycle-skipping. That is, it will only generate a new charge cycle at the next clock pulse if the secondary current is nearly zero, otherwise the cycle is skipped.

The disclosed charging circuit operates so that as the capacitor charges, the time to consume the energy decreases. When cycle-skipping, the current draw on the power source never exceeds the maximum design limit.

The disclosed charging circuit protects against damage from a short-circuit on the primary side of the transformer by monitoring cycle by cycle current.

The disclosed charging circuit precludes core saturation due to built-up residual charge that may occur when the secondary is not completely discharged (a phenomenon called core-walking resulting from the buildup of residual charge) by creating a cycle-by-cycle current monitor that is used to control the charge cycle.

The disclosed charging circuit manages charging from one or more power source cells to optimize the ability to charge the capacitor. The disclosed charging circuit manages power source charging by determining what average current the power source can support. Such management is particularly important because the average current may change as the power source ages or depletes.

### BRIEF DESCRIPTION OF DRAWINGS

The drawings illustrate preferred embodiments of the Charging Circuit for an Automated External Defibrillator according to the disclosure. The reference numbers in the drawings are used consistently throughout. New reference numbers in FIG. 2 are given the 200 series numbers. Similarly, new reference numbers in each succeeding drawing are given a corresponding series number beginning with the figure number.

FIG. 1 is a logic diagram for a charging circuit.

FIG. 2 is an alternative logic diagram for a charging circuit.

FIG. 3 is a diagram for a charging circuit.

FIG. 4 is an exploded view of a defibrillator.

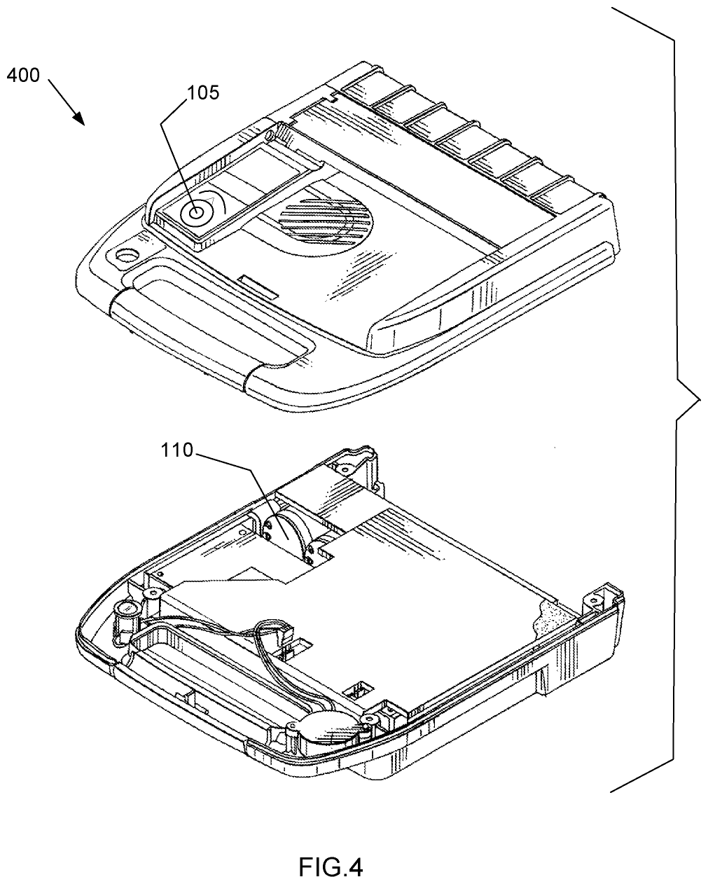

FIG. 5 is a charging circuit with optional components.

FIG. 6 is a charging circuit with a step-up/step down transformer coupled in a non-flyback-style circuit.

### DESCRIPTION OF EMBODIMENTS

In the following description, reference is made to the accompanying drawings, which form a part hereof and which illustrate several embodiments of the present invention. The drawings and the preferred embodiments of the invention are presented with the understanding that the present invention is susceptible of embodiments in many different forms and, therefore, other embodiments may be utilized, and structural, and operational changes may be made, without departing from the scope of the present invention.

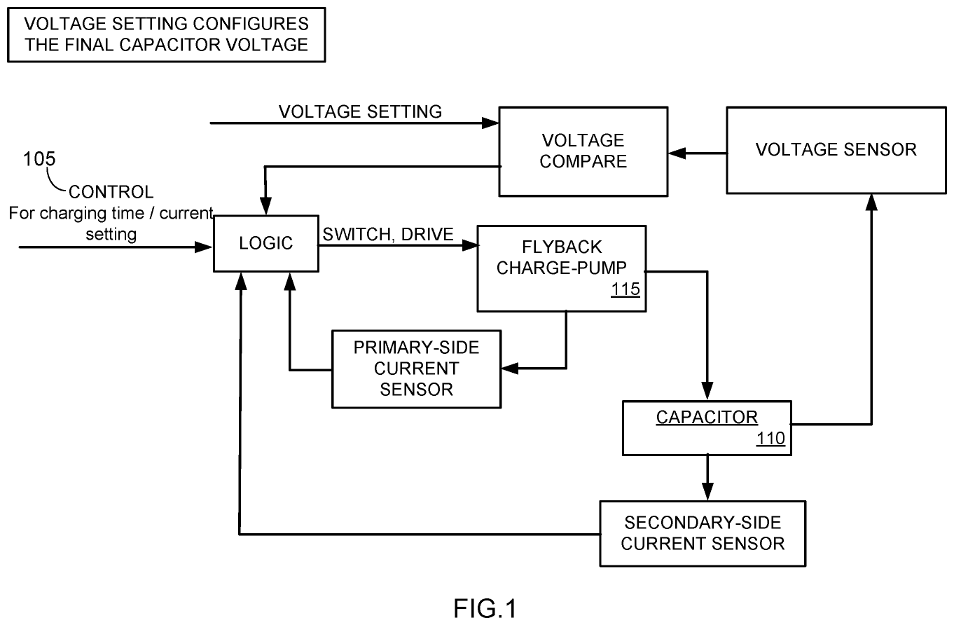

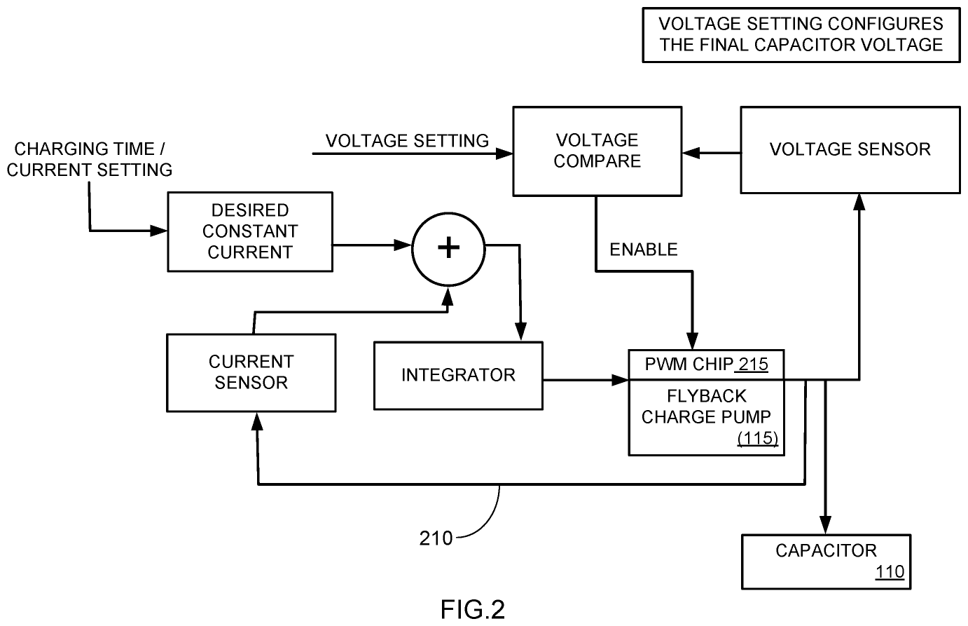

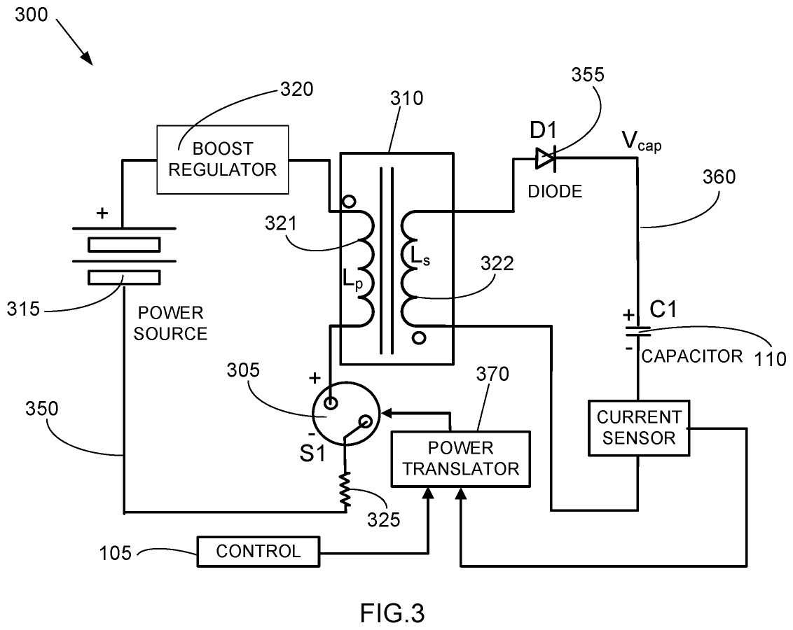

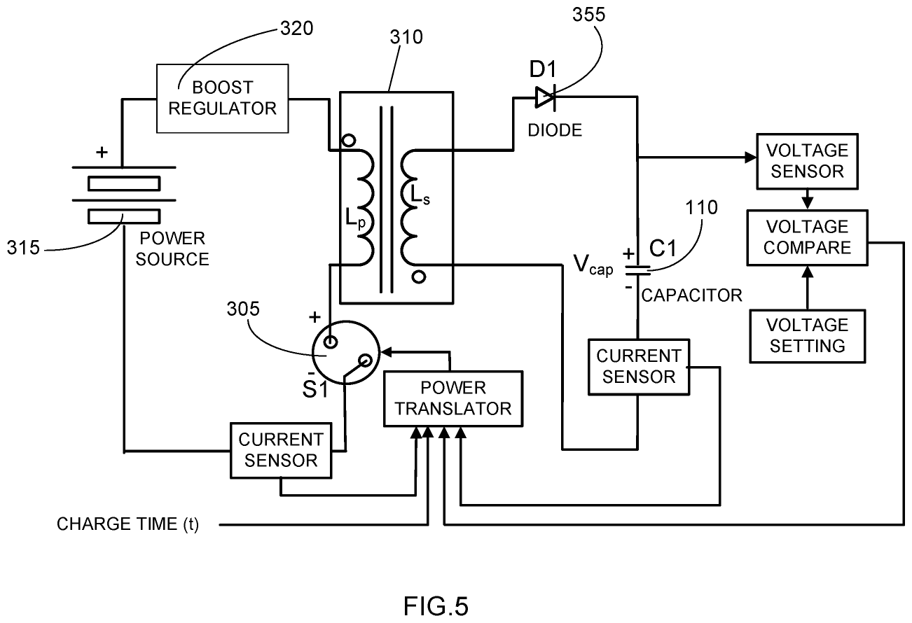

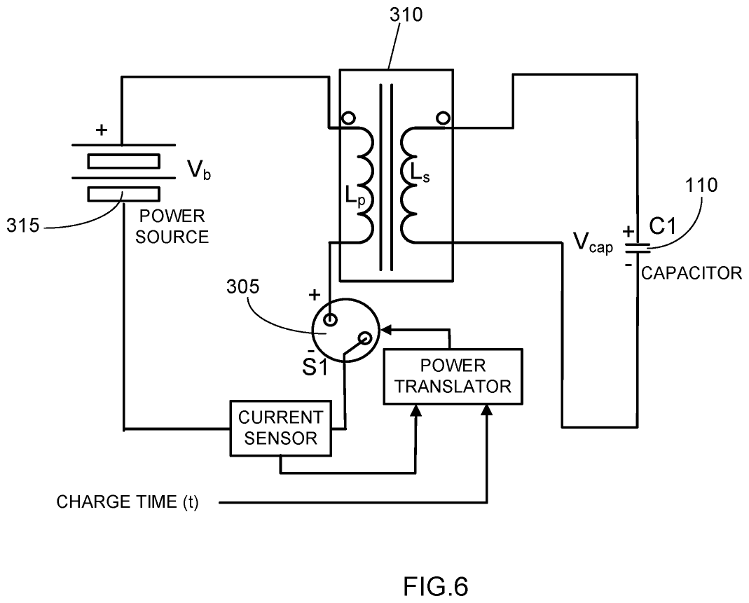

A charging circuit (300) for a capacitor (110) in a defibrillator (400) is described in a diagram in FIG. 1, FIG. 2, FIG. 3, FIG. 5 and FIG. 6. The charging circuit (300) includes a primary-side circuit (350); a secondary-side circuit (360); a control (105) enabling a setting of a desired time for charging a capacitor in the defibrillator (400); a transformer (310); a power source (315); a switch (305) in the primary-side circuit (350); and a power translator (370). The primary-side circuit (350) includes a primary-side induction coil (321) and the secondary-side circuit (360) includes a secondary-side induction coil (322).

The control (105) enables setting of a desired time for charging the capacitor (110) in the defibrillator (400). Preferably, the control (105) sets a duty cycle on the switch (305), thereby controlling the average current draw from the power source (315) to the primary-side induction coil (321). The duty cycle is the rate of opening and closing of the switch (305), which prevents or allows, respectively current flow in the primary-side circuit. The power translator (370) is configured to regulate the current draw on the power source (315) so that it is sufficient to enable charging the capacitor (110) within the desired time set on the control (105). An example of a control (105) is a software setting that will configure the charging time by varying the current from the power source (315) using this method.

The transformer (310) is electrically connected to the primary-side circuit (350). The switch (305) is configured to stop or enable current flow from the power source (315) to the primary-side circuit (350).

The primary-side circuit (350) and the secondary-side circuit (360) are galvanically isolated from each other. Galvanic isolation is a principle of isolating functional sections of the electrical circuits to prevent current flow between them, that is, such isolation eliminates any direct electrical conduction path between the circuits. There is a primary-side induction coil (321) is also designated Lₚ in FIG. 3, within the primary-side circuit (350). There is a secondary-side induction coil (322) is also designated Lₛ in FIG. 3, within the secondary-side circuit (360). The secondary-side induction coil (322) is configured to receive the magnetic energy input by the primary-side induction coil.

The power translator (370) is configured to vary a power draw from the power source (315) to meet the setting on the control (105) of the desired time for charging the capacitor (110). As a primary example, the power translator (370) controls the switch (305) to stop or enable current flow from the power source (315) to the primary-side induction coil (321) so that an average power draw from the power source (315) is sufficient to transfer energy to charge the capacitor (110) within the desired time set on the control (105).

### Example 1

In FIG. 1, the charging circuit (300) is shown to include a flyback charge-pump (115), which in this example is the power translator (370). The flyback-style circuit is also known as a “flyback transformer” made from a primary inductor and secondary inductor wound on a common core with the winding polarity 180 degrees out of phase to store energy for later release to a capacitor. In the example 1 charging circuit, the secondary side of the transformer has a switch, such as a diode (355), that prevents current flow from the capacitor back to the transformer when current flows in the primary induction coil. The transformer transfers its energy to a capacitor on the secondary side when the primary side switch is open. A flyback-style circuit, also referred to as the flyback charge-pump (115), has theoretically no limit on the output voltage from the charged capacitor and the transformer isolates and protects the primary-side circuitry from the secondary-side high-voltage circuitry by way of galvanic isolation.

The flyback charge-pump (115) may include a switch (305), which is configured to enable energy to be stored in the transformer (310). The transformer (310) is configured to transfer the energy to the capacitor (110). The flyback charge-pump (115) is preferably controlled by a duty-cycle on the switch (305) so that a current draw from a power source (315) is sufficient to enable charging the capacitor (110) within the desired time set on the control (105).

### Example 2

In FIG. 6, the charging circuit (300) is shown to include a step-up/step down transformer coupled in a non-flyback-style circuit. In this example, no energy is stored in the step-up/step down transformer. The non-flyback-style circuit eliminates the need for the diode (355), shown in FIG. 5, and the step-up/step down transformer has a primary-side induction coil (321) and secondary-side induction coil (322) that are not out of phase, that is, the primary and secondary winding polarities are in phase. Unlike the flyback-style circuit of example 1, the primary-side induction coil (321) and secondary-side induction coil (322) are not 180 degrees out of phase. So, in the charging circuit (300) of FIG. 6, energy is not stored in the transformer but instead, is immediately transferred to the capacitor (110).

FIG. 6 illustrates the non-flyback style circuit where there is no diode, the polarity of the transformer windings is “in-phase” and there is no storage of energy in the transformer. An operating characteristic of the FIG. 6 circuit is that there is a maximum output voltage. Basically, for each duty cycle:

V_{cap}=V_{b}*TR

where V_{cap} is the voltage at the capacitor, V_{b} is the battery voltage, and TR is the turns ratio, which equals the number of secondary turns divided by the number of primary turns.

If V_{cap} is greater than V_{b}, then the transformer is a step-up transformer. In the unlikely case where V_{cap} is less than V_{b}, then the transformer is a step-down transformer.

For the non-flyback style circuit of FIG. 6, no energy stored in the transformer. V_{cap} is limited to V_{b} times the turns ratio. There is no secondary current sensor needed and transformer saturation due to core-walking is not an issue.

FIG. 3 illustrates a flyback charge-pump (115) shown with a boost regulator (320). The boost regulator (320) is optional. The initial state is that the capacitor (110) is discharged, and the transformer has zero stored energy.

The boost regulator (320) is also shown in FIG. 5 with optional components, including a primary induction coil current sensor and a secondary winding current sensor. This circuit configuration configures the switch control to provide primary induction coil over-current protection. The current leaving the boost regulator (320), I_{primary}, is determined by the equation—

$${I}_{primary} = \frac{{V}_{b o o s t}}{{L}_{p}} * \frac{1}{2} * \frac{{DutyCycle}^{2}}{Frequency}$$

where V_{boost} is the boosted voltage from the boost regulator; Lₚ is the inductance of the primary induction coil; the DutyCycle is the time the switch is closed divided by the total cycle period (on time+off time); and the Frequency is the rate that cycle periods occur. The time, t, for charging the capacitor (110) is determined by the equation—

$$t = \frac{{V}_{c a p}^{2} * C}{{V}_{b} * {I}_{primary} * 2}$$

where V_{cap} is the voltage across the capacitor (110); V_{b} is the power source voltage. While the current sensor for the primary induction coil may be omitted, this would leave only the current sensor for the secondary winding. If only the secondary winding current sensor is included in the circuit, then there would be no over-current protection for the primary induction coil.

### Example 3

A circuit is configured as shown in FIG. 3. The flyback charge-pump (115) uses the flyback switch-mode power supply topology, and functions as follows: Switch (305), also labeled 51 in FIG. 3, is initially in the “OFF” position and no current flows into the primary induction coil of the transformer (310), also labeled Lₚ in FIG. 3. When the charging cycle for the flyback charge-pump (115) is activated by moving switch 51 to the “ON” position, the switch (305) then permits electrical current to flow into the primary induction coil Lₚ of the transformer (310) where energy is stored in the core for transfer to the secondary winding, labeled Lₛ in FIG. 3, in the transformer (310).

Because of the winding polarity and the presence of a diode (355), also labeled (D1), on the secondary side of the transformer (310), no energy is transferred (current cannot flow from the capacitor (110) towards the secondary winding Lₛ). Rather, energy is stored in a primary induction coil magnetic field of the transformer (310). When the switch (305) is changed to the “OFF” position, all the stored energy flows from the transformer (310) through the diode (355) on the secondary side and then into the capacitor (110). The voltage at Cl increases until all the energy transfers out of the transformer (310). The charging cycle is repeated until voltage (V_{cap}) reaches desired voltage setting.

### Example 4

A circuit is configured as shown in FIG. 1. The charging circuit (300) logic shown in FIG. 1 illustrates a configuration that maintains an approximately constant power from the power source (315) to the flyback charge-pump (115). The flyback charge-pump (115) is controlled so that it provides a constant average current draw on the power source (315). Being able to set the charging current enables determining the charging time. Regardless of the charge-pump implementation, given that the voltage (V_{bat}) to the primary induction coil in the transformer (310) is constant and the average current draw is also constant, then the time-to-charge the capacitor (110) follows this equation:

$$t = \frac{{V}_{cap}^{2} * C}{{V}_{i n} * {I}_{i n} * 2}$$

where t equals time; V_{cap} is the voltage acting on the capacitor (110); Vᵢₙ is the voltage to the primary induction coil of the transformer (310); and Iᵢₙ is the average charging current from the power source.

The charging circuit (300) may be further configured to monitor a cycle-by-cycle current to the capacitor (110) from the transformer (310), and to stop energy saturation of the transformer (310) by preventing the flyback charge-pump (115) from sending a charge pulse of energy to the transformer (310) unless the cycle-by-cycle current to the capacitor (110) is approximately zero.

The charging circuit (300) may be further configured to monitor a cycle-by-cycle current from the power source (315) to the transformer (310), and to stop energy draw from the power source (315) if an over-current is detected.

The charging circuit (300) may include a boost regulator (320) configured to deliver an approximately constant voltage to the charge pump. The boost regulator (320) is used to provide a known constant voltage to transformer (310). When the duty-cycle is constant, the average current, I_{primary}, leaving the boost regulator (320) for the primary induction coil is also known, and, then, so is charge time. The power draw on power source (315) is constant, even though voltage and current are not. Power source (315) health is maintained by controlling the power drawn. This is done by controlling I_{primary}, and therefore power stored by primary induction coil in the transformer (310), and therefore power transferred from the power source (315). As given above the current, I_{primary}, and charging time, t, are determined by the equations—

$${I}_{primary} = \frac{{V}_{b o o s t}}{{L}_{p}} * \frac{1}{2} * \frac{{DutyCycle}^{2}}{Frequency}$$

$$t = \frac{{V}_{c a p}^{2} * C}{{V}_{i n} * {I}_{i n} * 2}$$

The charging circuit (300) may include a variable resistor (325) configured to adjust its resistance in a feedback loop (210) to maintain an approximately constant average charging current delivered to the capacitor (110).

Varying or controlling the duty cycle of the switch (305) can create a constant average current source. Given a fixed power source voltage, the equation for the current draw is:

$$I = \frac{{V}_{i n}}{{L}_{p}} * \frac{1}{2} * \frac{{DutyCycle}^{2}}{Frequency}$$

where I is the average current draw from the power source (315); Vᵢₙ is the voltage to the primary induction coil of the transformer (310); Lₚ is the inductance of the primary induction coil.

So, controlling the duty cycle sets the average current draw, and therefore sets the charging time.

The charging circuit (300) may be further configured to vary a duty cycle of the switch (305) to maintain a constant average current from the power source (315) where voltage is made constant with a boost regulator (320).

The charging circuit (300) may be further configured to vary a duty cycle of the switch (305) to update the constant average current from the power source (315) as a function of any change in a voltage at the power source (315), maintaining a constant power to the charging circuit.

The charging circuit (300) may be further configured to vary an on or off duty cycle of the switch (305) to maintain a constant average current from the power source (315).

To avoid transformer (310) saturation in this configuration, the charging circuit (300) would then monitor the current on the secondary side of the charge-pump on every charging cycle. On the primary side, this configuration may include a cycle-by-cycle current monitor, which prevents two problems: damage from a short-circuit on the primary, and core saturation due to built-up residual charge.

Essentially, this charging circuit (300) configuration would not allow the next cycle to begin until the current transferred from the transformer has dropped to nearly zero. This assures that all the energy stored in the transformer will have been consumed.

Given a fixed frequency, the operation will appear to cause cycle-skipping because a new charge pulse would only generate at the next clock pulse if the secondary current is nearly zero, otherwise the cycle would be skipped. As the capacitor (110) charges, the time to consume the energy decreases because it follows the principle of Volt-second conservation. So as the capacitor (110) charges up, it takes less time to consume the energy, and soon there are no more cycle-skipping events. When cycle-skipping, the current draw on the power source never exceeds the maximum design limit.

For the charging circuit (300) configuration diagrammed in FIG. 1, the equation for the charging time for the capacitor (110) is

$$t = \frac{{V}_{o u t}^{2} * C}{{V}_{i n} * {I}_{i n} * 2}$$

where t is the charging time; Vₒᵤₜ is the desired output voltage; C is the capacitance; Iᵢₙ is the desired constant average current, and Vᵢₙ is the input voltage. When C and Vᵢₙ are constants, adjusting the current setting results in a known charge time. In this configuration, Iᵢₙ is a function of the duty cycle.

For the charging circuit (300) configuration diagrammed in FIG. 1, the equation for the charging time for the capacitor (110) can be derived from the above equation as follows since is current multiplied by voltage equals power Pᵢₙ.

$$t = \frac{{V}_{o u t}^{2} * C}{{P}_{i n} * 2}$$

The charging circuit (300) may be further configured to vary a duty cycle of the switch (305) to maintain a constant average current from the power source (315) as a function of an inductance of the primary induction coil in the transformer (310).

The charging circuit (300) may include a pulse-width modulation chip (215) configured to sense cycle-by-cycle changes in current draw from the power source (315) and respond by adjusting a variable resistor (325) to maintain a constant average current draw from the power source (315).

The pulse-width modulation chip (215) creates a flyback implementation modified to provide a constant average current draw from the power source to charge the capacitor (110). The standard pulse-width modulation chip does not provide a constant average current source. However, this implementation is different. This implementation converts the pulse-width modulation chip (215) into a variable average current source that can also control startup transients, i.e., charging at startup. This implementation is shown in FIG. 2, which is characterized by a feedback loop with integration control. The pulse-width modulation chip (215) is combined with the flyback charge-pump (115). Everything else shown in FIG. 2 is used to control the combined pulse-width modulation chip (215) and flyback charge-pump (115). The implementation in FIG. 2 limits the average charging current equal to the current set by a user at the outset of the charging cycle.

The charging circuit may be configured to monitor capacitor leakage to maintain a specified voltage acting on the capacitor (110).

Over-current protection on the primary side is monitored cycle-by-cycle by the pulse-width modulation chip (215). Traditional pulse-width modulation (PWM) chips have threshold detectors but the pulse-width modulation chip (215) used herein is different because a variable resistor (325) which is a variable current sensing resistor is added. Additionally, the charging circuit (300) uses a feedback loop to control that resistor. This variable resistor (325) is set to a value that is a function of the average power source current. Where the pulse-width modulation chip (215) is operating cycle by cycle, the variable resistor (325) is controlling the pulse-width modulation chip (215). This variable resistor (325) is in turn controlled (through the feedback loop) by a circuit that detects the average current. The capacitor output voltage is monitored and controlled by the pulse-width modulation chip (215), also labeled PWN in FIG. 2. The combined pulse-width modulation chip (215) and flyback charge-pump (115) may be used to function as a built-in switch driver.

The above-described embodiments including the drawings are examples of the invention and merely provide illustrations of the invention. Other embodiments will be obvious to those skilled in the art. Thus, the scope of the invention is determined by the appended claims and their legal equivalents rather than by the examples given.

### INDUSTRIAL APPLICABILITY

The invention has application to the emergency rescue industry.

What is claimed is:

1. A charging circuit for a capacitor in a defibrillator, the charging circuit comprising:
a control enabling a setting of a desired time for charging a capacitor in the defibrillator;
a transformer comprising:
a primary-side circuit and a secondary-side circuit, the secondary-side circuit galvanically isolated from the primary-side circuit;
the primary-side circuit comprising a primary-side induction coil; and
the secondary-side circuit comprising a secondary-side induction coil configured to receive the magnetic energy input by the primary-side induction coil;
a power source electrically connected to the primary-side circuit; and
a power translator configured to vary a power draw from the power source to meet the setting on the control of the desired time for charging the capacitor.

2. The charging circuit of claim 1, further comprising a switch in the primary-side circuit, the switch configured to stop or enable current flow from the power source to the primary-side circuit, wherein the power translator controls the switch to stop or enable current flow from the power source to the primary-side induction coil so that an average power draw from the power source is sufficient to transfer energy to charge the capacitor within the desired time set on the control.

3. The charging circuit of claim 1, wherein the power translator comprises a flyback-style circuit configured to have no limit on output voltage from a charged capacitor.

4. The charging circuit of claim 1, wherein the power translator comprises a step-up/step down transformer coupled in a non-flyback-style circuit.

5. The charging circuit of claim 1, configured to maintain an approximately constant average power from the power source to the power translator.

6. The charging circuit of claim 1, configured to monitor a cycle-by-cycle current to the capacitor from the transformer, and to stop energy saturation of the transformer by preventing power translator from energizing the transformer unless the cycle-by-cycle current to the capacitor is approximately zero.

7. The charging circuit of claim 1 configured to monitor a cycle-by-cycle current from the power source to the transformer, and to stop energy draw from the power source if an over-current is detected.

8. The charging circuit of claim 1, further comprising a boost regulator configured to deliver an approximately constant voltage to the primary-side induction coil in the transformer.

9. The charging circuit of claim 1, further comprising a variable resistor configured to adjust its resistance in a feedback loop to maintain an approximately constant average charging current delivered to the capacitor.

10. The charging circuit of claim 1, further comprising a pulse-width modulation chip configured to sense cycle-by-cycle changes in current draw from the power source and respond by adjusting the variable resistor to maintain a constant average current to the primary-side induction coil of the transformer.

11. The charging circuit of claim 1, further comprising a boost regulator, the charging circuit configured to vary a duty cycle of the switch to maintain a constant average current to the primary-side induction coil in the transformer where a boost regulator is configured to provide a constant voltage to the transformer.

12. The charging circuit of claim 1 configured to vary a duty cycle of the switch to update a constant average current from the power source as a function of any change in a voltage across the power source.

13. The charging circuit of claim 1 configured to vary a duty cycle of the switch to maintain a constant average current from the power source as a function of an inductance of a primary induction coil in the transformer.

14. The charging circuit of claim 1 configured to monitor capacitor leakage to maintain a specified voltage across the capacitor.
